(19) United States
(12) Patent Application Publication
(10) Pub. No.: US 2013/0304000 A1
(43) Pub. Date: Nov. 14, 2013

(54) BODY PIERCING CLEANSING AND SANITATION DEVICE

(71) Applicant: Christian James Pentell, Palos Park, IL (US)

(72) Inventor: Christian James Pentell, Palos Park, IL (US)

(21) Appl. No.: 13/893,544

(22) Filed: May 14, 2013

Related U.S. Application Data
(60) Provisional application No. 61/646,457, filed on May 14, 2012.

(51) Int. Cl. A61M 35/00

(52) U.S. Cl. CPC A61M 35/00
USPC 604/289

(57) ABSTRACT

A body piercing cleansing and sanitation device is described, which is self-contained and pressurized. The device improves and simplifies the cleaning process, ensuring thorough sanitation through the piercing hole, the external surface of the piercing, and the body ring without needing to remove the body ring.

### CROSS-REFERENCE TO RELATED APPLICATIONS

This application claims benefit under 35 U.S.C. §119(e) of U.S. Provisional Patent Application Ser. No. 61/646,457, entitled “BODY PIERCING CLEANSING AND SANITATION DEVICE”, filed May 14, 2012, naming Christian J. Pentell as the inventor, the complete disclosure being incorporated herein by reference.

### BACKGROUND

The present application generally relates to a cleansing and sanitation device for cleaning a body piercing. Body piercing is a common practice. Generally, body piercing consists of puncturing a small hole through the skin of a body part, such as an ear, nose, tongue or navel. A body ring, such as a decorative accessory or piece of jewelry, is then typically inserted into the piercing.

Because a piercing is essentially an open wound, it is important to keep the piercing clean to avoid infection. In general, piercing hygiene traditionally includes removing the body ring and applying a cleaning solution to the exterior surface of the piercing. For example, this may include removing an earring to allow a cotton swab containing cleaning solution to pass over the surface of an ear piercing. Other methods include inserting a cleansing tool, such as a sterile string or thread, through the piercing to clean the inside thereof. For example, U.S. patent application No. 11/614,600 (Zocher) is directed to a cleaning apparatus for a body piercing including a cleaning solution applicator that is pulled through the body piercing for cleansing thereof. Similarly, U.S. Pat. No. 6,042,572 issued to Fowler, is directed to a hygiene device for cleaning a body passageway formed within a piercing by inserting a cleaning solution application into the body piercing.

However, a new piercing requires the body ring (i.e., earring) to remain in the piercing for a specific amount of time, such as 4-6 weeks, to avoid the piercing from closing due to the body's natural healing process. Additionally, it is generally necessary to clean new piercings at least 2-3 times daily to avoid infection. The conventional swabbing method described above is often ineffective for new piercings because access to the piercing is generally impaired by the body ring. Accordingly, the solvent applied to the exterior of the piercing may not be able to penetrate to the inside of the piercing. As a result, there is a serious risk of areas inside the piercing to remain unsanitized. Moreover, cleaning the inside of the piercing becomes particularly difficult as the piercing excretes blood, pus, and other bodily fluids that produce scabbing inside and around the piercing. As a result, it is an object of the present application to provide a body piercing cleaning device that effectively sanitizes new piercings, despite the presence of a body ring inside the piercing.

Specifically, it is an object of the present application to provide a body piercing cleansing and sanitation device, which is self-contained and pressurized, that improves and simplifies the cleaning process, ensuring thorough sanitation through the piercing hole, the external surface of the piercing, and the body ring without needing to remove the body ring.

### SUMMARY OF THE DISCLOSURE

In accordance with the present application, provided is a body piercing cleansing and sanitation device, which is self-contained and pressurized, that improves and simplifies the cleaning process, ensuring thorough sanitation through the piercing hole, the external surface of the piercing, and the body ring without needing to remove the body ring.

### BRIEF DESCRIPTION OF THE DRAWINGS

FIG. 1 is a cross-sectional view of a first embodiment of the present body piercing cleaning and sanitation device.

FIG. 2 is a cross-sectional view of a second embodiment of the present body piercing cleaning and sanitation device.

FIG. 3 is a cross-sectional view of a third embodiment of the present body piercing cleaning and sanitation device.

FIG. 4 is a cross-sectional view of a fourth embodiment of the present body piercing cleaning and sanitation device.

### DETAILED DESCRIPTION OF THE DRAWINGS

Embodiments of the present application relate to a body piercing cleansing and sanitation device for cleaning a new piercing containing a body ring therein. The following description is presented to enable one of ordinary skill in the art to make and use the device and is provided in the context of a patent application and its requirements. Various modifications to the preferred embodiment and the generic principles and features described herein will be readily apparent to those skilled in the art. Thus, the present application is not intended to be limited to the embodiments shown, but is to be accorded the widest scope consistent with the principles and features described herein. Advantages of the present device will now be described in detail with references to the accompanying drawings.

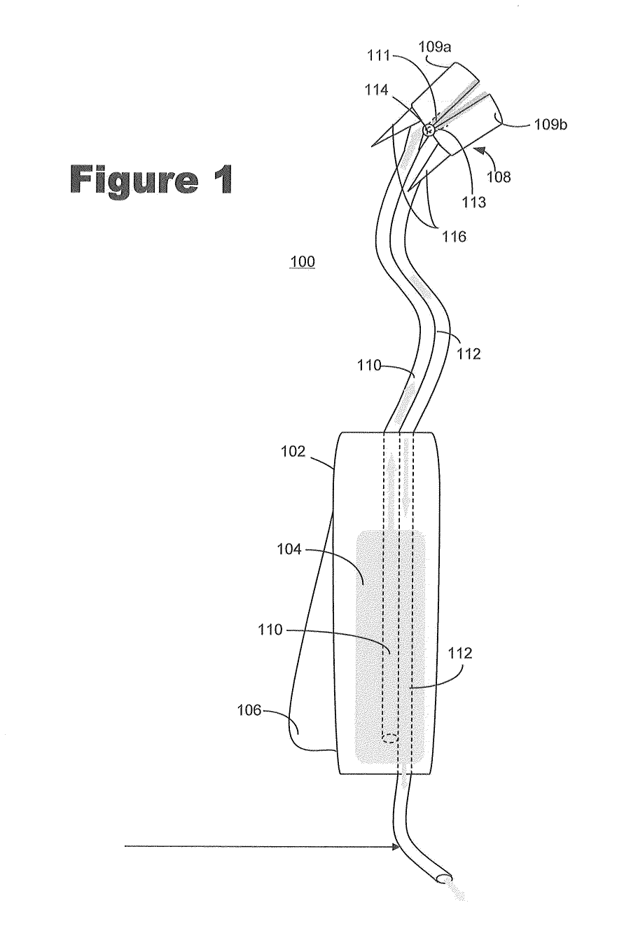

FIG. 1 is a cross-sectional view of a body cleansing and sanitation device 100 according to a first embodiment of the present application. The present device 100 is comprised of body portion 102 containing a reservoir 104, an enclosable cleansing chamber 108, a pressure pump 106, and a plurality of tubing 110, 112 therein. Specifically, the reservoir 104 is comprised of a large cavity defined inside the body portion 102 for containing liquid, namely cleaning solution, solvent or the like, therein. The reservoir 104 is in fluid communication with an enclosable cleansing chamber 108 via tubing. Specifically, the enclosable cleansing chamber 108 consists of two dome-shaped halves 109a, 109b, each defining an aperture 111, 113, respectively therein for receiving liquid from delivery tube 110 and expelling liquid from drainage tube 112, respectively.

The halves 109a, 109b of the enclosable cleansing chamber 108 converge at one end by means of a hinge 114 (or similar fastening mechanism) and extend into two spring-loaded levers 116, which actuate the hinged fulcrum point 114. The spring-loaded levers 116 are squeezed together, thus opening the enclosable cleansing chamber 108 and allowing insertion of a body part, such as an ear lobe, and clamping onto the body part upon releasing the levers 116. This creates a tightly sealed and drip-proof enclosure allowing the solution to maintain pressure. In one embodiment, the surface area defined by the enclosable cleansing chamber 108 is sized and shaped to enclose or otherwise envelope a body ring situated in relation to the pierced body part. In another embodiment, the areas of the enclosable cleansing chamber 108 which contact the body part may be formed of rubber, foam or the like to provide for an improved sealing surface therebetween

Generally, liquid is housed in the reservoir 104 until a pressure pump 106 is actuated to apply pressure to the liquid, causing it to flow from the reservoir 104, through the delivery tube 110, and into one half 109a of the enclosable cleansing chamber 108 via the aperture 111. When the enclosable cleansing chamber 108 is sealed about a pierced body part, the liquid irrigates or otherwise cleanses that pierced body part along with any body ring of which the enclosable cleansing chamber 108 envelops. The aperture 113 defined in the enclosable cleansing chamber 108 allows liquid to evacuate through the other half 109b of the chamber 108 via a drainage tube 112 and out of the device into a drainage collection receptacle (not shown), sink (not shown) or other such waste receptacle. In one embodiment, the pressure pump 106 is actuated by squeezing a handle. Alternatively, the pressure pump may be motor activated (not shown).

Specifically, the present pressure pump provides a pressurized rinsing feature to force the cleaning solution through the pierce-hole as well as the surrounding surface. This is particularly advantageous because the pierce-hole generally bleeds for several days, causing scabbing around and within the pierce-hole, making cleaning difficult. In the present embodiment, when scabbing exists, the positively pressurized applicator distributes the force necessary to rinse the scabbing out of the pierce-hole as well as over the surrounding surface of the pierce-hole. As a result, the risk of infection is significantly reduced.

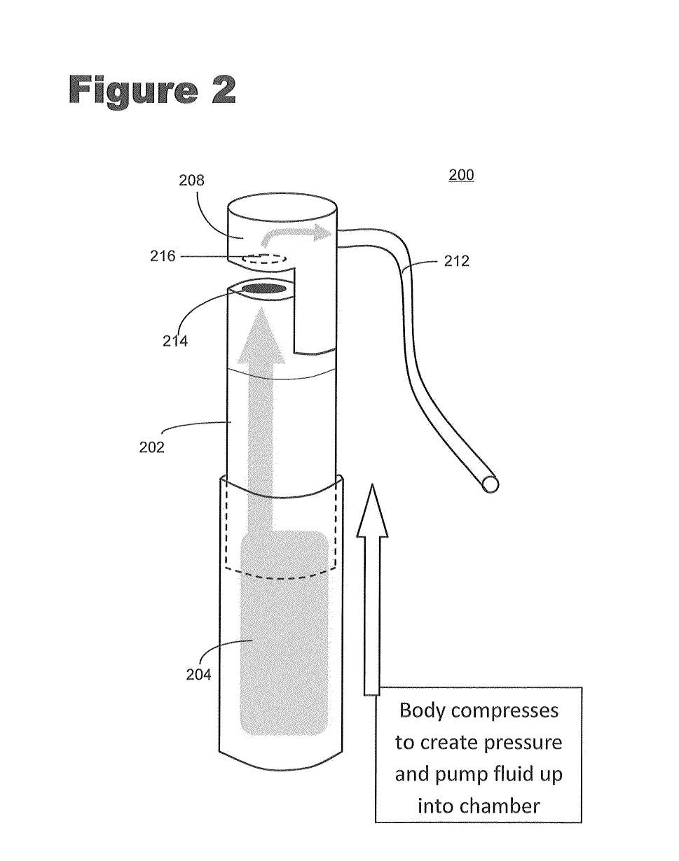

In another embodiment, illustrated in FIG. 2, the present device 200 is comprised of body portion 202 containing a reservoir 204, an enclosable cleansing chamber 208, a pressure pump 206, and a plurality of tubing 210 therein. Specifically, the reservoir 204 is comprised of a large cavity defined inside the body portion 202 for containing liquid, namely cleaning solution, therein. In this embodiment, the body portion 202 includes a telescoping arrangement to allow the body portion 202 to compress to create pressure within the reservoir 204. The top area of the body portion 202 defines an aperture 214 therein, and the bottom portion of the chamber 208 defines an aperture 216 therein to allow fluid communication therebetween. Generally, liquid is housed in the reservoir 204 until the body portion 202 is depressed to apply pressure to the liquid, causing it to flow from the reservoir 204, through the apertures 214, 216, and into the enclosable cleansing chamber 208. When the enclosable cleansing chamber 208 is sealed about a pierced body part, the liquid irrigates or otherwise cleanses that pierced body part along with any body ring of which the enclosable cleansing chamber 208 envelops. The drainage tube 212 allows the solution to evacuate through the chamber 208 via a drainage tube 212 out of the device 200.

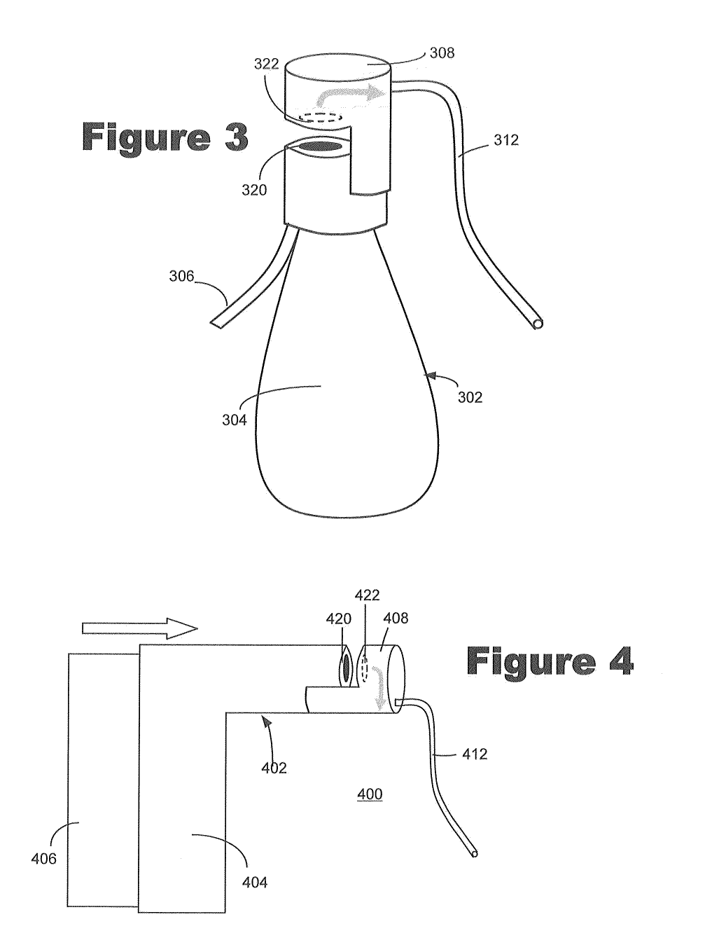

FIG. 3 is a cross-sectional view of a body cleansing and sanitation device 300 according to a third embodiment of the present application. The present device 300 is comprised of body portion 302 containing a reservoir 304, an enclosable cleansing chamber 308, a pressure pump 306, and a draining tube 312. Specifically, the reservoir 304 is comprised of a large cavity defined inside the body portion 302 for containing liquid, namely cleaning solution, therein. The reservoir 304 is in fluid communication with an enclosable cleansing chamber 308 via tubing. Specifically, the chamber 308 defines an aperture 322 therein for receiving liquid from the body portion via the aperture 320 defined therein. Generally, liquid is housed in the reservoir 304 until a pressure pump 306 is actuated to apply pressure to the liquid, causing it to flow from the reservoir 304 to the enclosable cleansing chamber 308 via apertures 320, 322. There is a space defined between the body portion 320 and the enclosable cleansing chamber 308 to allow insertion of a body part therebetween, such as an ear lobe, and clamping onto the body part. This creates a tightly sealed and drip-proof enclosure allowing the solution to maintain pressure. When the enclosable cleansing chamber 308 is sealed about a pierced body part, the liquid irrigates or otherwise cleanses that pierced body part along with any body ring of which the enclosable cleansing chamber 308 envelops. The drainage tube 312 allows the solution to evacuate through one end of the chamber 308 via a drainage tube 312 out of the device. In one embodiment, the pressure pump 306 is actuated by squeezing a handle.

FIG. 4 is a cross-sectional view of a body cleansing and sanitation device 400 according to a fourth embodiment of the present application. The present device 400 is comprised of body portion 402 containing a reservoir 404, an enclosable cleansing chamber 408, a pressure pump 406, and a draining tube 412. Specifically, the reservoir 404 is comprised of a large cavity defined inside the body portion 402 for containing liquid, namely cleaning solution, therein. The reservoir 404 is in fluid communication with an enclosable cleansing chamber 408 via tubing. Specifically, the chamber 408 defines an aperture 422 therein for receiving liquid from the body portion via the aperture 420 defined therein. Generally, liquid is housed in the reservoir 404 until a pressure pump 406 is actuated to apply pressure to the liquid, causing it to flow from the reservoir 404 to the enclosable cleansing chamber 408 via apertures 420, 422. There is a space defined between the body portion 420 and the chamber 408 to allow insertion of a body part therebetween, such as an ear lobe, and clamping onto the body part. This creates a tightly sealed and drip-proof enclosure allowing the solution to maintain pressure. When the enclosable cleansing chamber 408 is sealed about a pierced body part, the liquid irrigates or otherwise cleanses that pierced body part along with any body ring of which the enclosable cleansing chamber 408 envelops. The drainage tube 412 allows the solution to evacuate through one end of the chamber 408 via a drainage tube 412 out of the device. In one embodiment, the pressure pump 406 is actuated by squeezing a handle.

Various modifications to the preferred embodiments of the body piercing cleaning and sanitation device and the generic principles and features described above will be readily apparent to those skilled in the art. Thus, the body piercing cleansing and sanitation device is not intended to be limited to the embodiments shown, but is to be accorded the widest scope consistent with the principles and features described above.

1. A device for cleansing and sanitizing a pierced body part, comprising:
a reservoir for containing cleaning solution, and
an enclosable cleansing chamber in fluid communication with the reservoir and adapted to receive and expel the cleaning solution, wherein the cleansing chamber is adapted to envelop the pierced body part and thereby clean the pierced body part when the cleaning solution is received and expelled from the cleansing chamber.

2. The device of claim 1, wherein the cleansing chamber is further adapted to envelop a piercing situated in relation to the pierced body part.

3. The device of claim 1, wherein the cleansing chamber further comprises two dome shaped halves which are closable to envelop the pierced body part.

4. The device of claim 3, wherein the dome shaped halves are hinged at one end and thereby closable to envelop the pierced body part.

5. The device of claim 1, wherein the cleansing chamber further comprises a sealing surface for engagement between the sealing surface and the pierced body part.

6. The device of claim 1, wherein the fluid communication between the reservoir and the enclosable cleansing chamber is pressurized.

7. The device of claim 7, further comprising a pressure pump for pressurizing the fluid communication between the reservoir and the enclosing cleansing chamber.
